(19) United States
(12) Patent Application Publication
(10) Pub. No.: US 2021/0375441 A1
(43) Pub. Date: Dec. 2, 2021

(54) USING CLINICAL NOTES FOR ICU MANAGEMENT

(71) Applicants: Shafiq Rayhan Joty, Singapore (SG); Regents of the University of Minnesota, Minneapolis, MN (US)

(72) Inventors: Karan Aggarwal, Seattle, WA (US); Swaraj Khadanga, San Jose, CA (US); Shafiq Rayhan Joty, Singapore (SG); Jaideep Srivastava, Plymouth, MN (US)

(21) Appl. No.: 17/330,908

(22) Filed: May 26, 2021

Related U.S. Application Data
(60) Provisional application No. 63/032,016, filed on May 29, 2020.

(51) Int. Cl. G16H 40/20; G16H 40/67; G16H 10/60; G16H 50/70; G16H 50/20; G06N 3/04; G06F 40/40

(52) U.S. Cl. CPC G16H 40/20; G16H 40/67; G16H 10/60; G06F 40/40; G16H 50/20; G06N 3/0454; G06N 3/0445; G16H 50/70

(57) ABSTRACT

A method can be implemented at one or more computing machines. The method can include receiving, using a server, time-series data corresponding to monitoring instrumentation in a medical care facility. The time-series data corresponds to a selected care recipient. The time-series data is stored in one or more data storage units. The time-series data includes data correlated with a plurality of regular time intervals. The method includes receiving, using a server, aperiodic data corresponding to clinical notes collected in the medical care facility and corresponding to the selected care recipient. The aperiodic data is stored in one or more data storage units. The aperiodic data includes a time stamp. The method includes generating, using a deep neural network and the time-series data and using a convolutional neural network (CNN) and the aperiodic data, a plurality of computer-generated data corresponding to management of the medical care facility or medical condition of the care recipient.

### CROSS-REFERENCE TO RELATED APPLICATIONS

This application claims the benefit of the filing date of U.S. Provisional Application Ser. No. 63/032,016, filed on May 29, 2020, the disclosure of which is incorporated by reference herein.

### TECHNICAL FIELD

This document pertains generally, but not by way of limitation, to processing clinical notes and time-series data for ICU management.

### BACKGROUND

Health care services are typically rather costly, and the specialized services provided in an intensive care unit are substantially more costly. Some forces driving the high cost include monitoring equipment and sophisticated technology used for treating specific medical conditions. In addition, the medical personnel working in the ICU and in support roles are highly trained and generally well-paid.

The following publications may provide context for selected aspects of the subject matter:
1. Hrayr Harutyunyan, Hrant Khachatrian, David C Kale, Greg Ver Steeg, and Aram Galstyan. 2017. Multitask Learning and Benchmarking with Clinical Time Series Data. arXiv preprint arXiv:1703.07771.
2. Simon Baker, Anna Korhonen, and Sampo Pyysalo. 2016. Cancer Hallmark Text Classification Using Convolutional Neural Networks. In Proceedings of the Fifth Workshop on Building and Evaluating Resources for Biomedical Text Mining (BioTxtM2016), pages 1-9.
3. Harini Suresh, Jen J Gong, and John V Guttag. 2018. Learning Tasks for Multitask Learning: Heterogenous Patient Populations in the ICU. In Proceedings of the 24th ACM SIGKDD International Conference on Knowledge Discovery & Data Mining, July 2018; pages 802-810. ACM.
4. Mengqi Jin, Mohammad Taha Bahadori, Aaron Co-lak, Parminder Bhatia, Busra Celikkaya, RamBhakta, Selvan Senthivel, Mohammed Khalilia, Daniel Navarro, Borui Zhang, et al. 2018. Improving Hospital Mortality Prediction with Medical Named Entities and Multimodal Learning.arXivpreprint arXiv:1811.12276.

### SUMMARY

In view of the challenges associated with monitoring ICU patients, an example of the present solution provides a solution that can help provide better acute care and assist in planning for the allocation of hospital resources for purposes of delivering better outcomes. In one example, the present subject matter can help predict the condition of patients over the course of their time in the ICU.

One example of the present subject matter provides machine learning for improving ICU management. Patient data can include time-series signals recorded by ICU instruments and can include clinical notes.

In evaluating efficiency for managing the ICU, three benchmarks can be considered. Suitable benchmarks can include in-hospital mortality prediction, modeling decompensation, and length of stay forecasting. While the time-series data is measured at regular intervals, care-giver notes are charted at irregular times, making it challenging to model them together. One example of the present subject matter includes a method to model time-series data and aperiodic notes in joint, thus achieving improvement across selected benchmark tasks relative to a baseline of time-series data only.

The time-series data can be provided by medical instruments located, for example, in the ICU. Aperiodic notes can include expert knowledge, such as clinical notes from a doctor. The time-series data can be measured continuously, and the aperiodic notes can be charted at discrete, or intermittent, times. A multi-modal deep neural network can analyze recurrent units for the time-series and convolution network for the clinical notes.

Each of these non-limiting examples can stand on its own or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates ICU management based on data from doctor notes and measured physiological signals.

FIG. 2 illustrates a block diagram from the in-hospital mortality multi-modal network.

FIG. 3 illustrates a block diagram from decompensation and length of stay prediction multi-modal network.

FIG. 4 illustrates the training and use of a machine-learning program, according to some embodiments.

FIG. 5 illustrates an example neural network, in accordance with some embodiments.

FIG. 6 illustrates the training of a machine learning program, in accordance with some embodiments.

FIG. 7 illustrates the feature-extraction process and classifier training, according to some example embodiments.

FIG. 8 illustrates a circuit block diagram of a computing machine in accordance with some embodiments.

FIG. 9 illustrates an example system in which artificial intelligence is implemented.

FIG. 10 illustrates a graph showing decompensation, according to one example.

### DETAILED DESCRIPTION

With the advancement of medical technology, patients admitted into the intensive care unit (ICU) are monitored by different instruments on their bedside, which measure different vital signals about patient's health. During their stay, doctors visit the patient intermittently for check-ups and make clinical notes about the patient's health and physiological progress. These notes can be perceived as summarized expert knowledge about the patient's state. All these data about instrument readings, procedures, lab events, and clinical notes are recorded for reference.

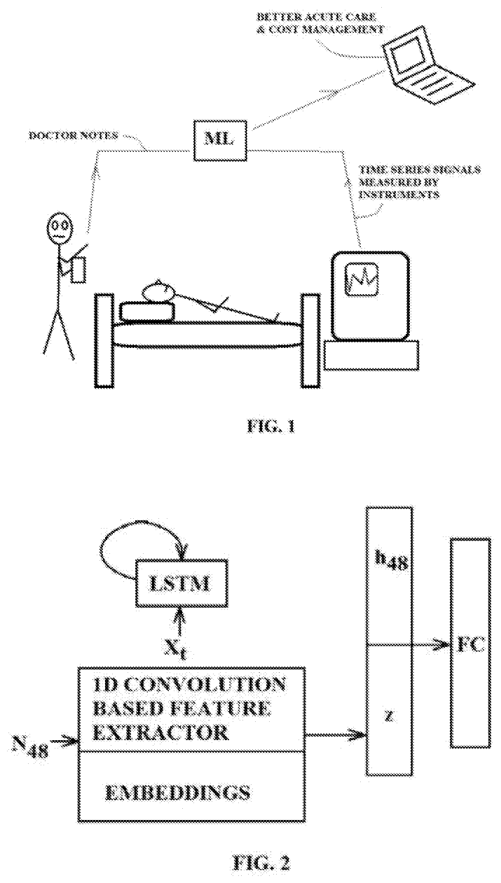

In one example, clinical notes and the time-series data are combined for improved prediction on benchmark ICU management tasks. The time-series data is measured continuously. The doctor notes are charted at intermittent times. One example includes a multimodal deep neural network that comprises of recurrent units for the time-series and convolution network for the clinical notes. The combination of clinical notes and time-series data improves the performance on metrics including in-hospital mortality prediction, modeling decompensation, and length of stay forecasting tasks. FIG. 1 illustrates ICU management based on data from doctor notes and measured physiological signals.

Biomedical natural language processing can be used in one example. Deep learning-based techniques for natural language processing can be used for clinical notes. Convolutional neural networks can be used to predict International Classification of Diseases (ICD) codes from clinical texts. In addition, a convolutional neural network can be used to classify various biomedical articles. Pre-trained word and sentence embeddings show good results with sentence similarity tasks.

Consider next, using clinical notes for ICU related tasks. Given the long-structured nature of clinical text, the convolutional neural network is preferred over recurrent networks. One example uses aggregated word embeddings (WE) of clinical notes for in-hospital mortality prediction.

ICU management related literature can be used in one example. ICU management can use time-series measurements for the prediction tasks. Recurrent neural networks (RNN) can provide a model for use with attention or multi-task learning. Supplemental information, like diagnosis, medications, lab events etc., can be used to improve model performance. One example uses RNNs for modeling time-series. Multi-modal learning can be used for speech, natural language, and computer vision. In addition, images/videos can be used with natural language text. In one example, clinical notes with time-series data can be used for ICU management tasks.

Consider three benchmarking tasks.

In-hospital Mortality refers to a binary classification problem to predict whether a patient dies before being discharged from the first two days of ICU data.

Decompensation concerns detecting patients who are physiologically declining. Decompensation is defined as a sequential prediction task where the model makes a prediction at each hour after ICU admission. The target at each hour is to predict the mortality of the patient within a 24-hour time window.

Length of Stay Forecasting (LOS) is a prediction of bucketed remaining ICU stay time with a multiclass classification problem. Remaining ICU stay time is discretized into 10 buckets: {0-1, 1-2, 2-3, 3-4, 4-5, 5-6, 6-7, 7-8, 8-14, 14+} days where the first bucket, covers the patients staying for less than a day (24 hours) in ICU and so on. This is only done for the patients that did not die in ICU.

These tasks correlate with performance indicators of models that can be beneficial in ICU management. In one example, RNN is used to model the temporal dependency of the instrument time series signals for these tasks.

Consider models by which the present subject matter can be used.

For a patient's length of ICU stay of T hours, consider a time-series of observations, xₜ at each time step t (1-hour interval) measured by instruments along with doctor's note nᵢ recorded at irregular time stamps. Formally, for each patient's ICU stay, a time series data [xₜ]ₜ₌₁^{T} of length T, and K doctor notes [Nᵢ]ᵢ₌₁^{K} are charted at time [TC(i)]ᵢ₌₁^{K}, where K is generally much smaller than T For in-hospital mortality prediction, m is a binary label at t=48 hours, which indicates whether the person dies in ICU before being discharged. For decompensation prediction performed hourly, [dₜ]ₜ₌₅^{T} are the binary labels at each time step t, which indicates whether the person dies in ICU within the next 24 hours. For LOS forecasting also performed hourly, [lₜ]ₜ₌₅^{T} are multi-class labels defined by buckets of the remaining length of stay of the patient in ICU. Use N_{T} to denote the concatenated doctor's note during the ICU stay of the patient (i.e., from t=1 to t=7).

### Time-Series LSTM Model

The baseline model can be evaluated with selected benchmark models. For all the three tasks, consider a Long Short Term Memory or LSTM network to model the temporal dependencies between the time series observations, [xₜ]ₜ₌₁^{T}. At each step, the LSTM composes the current input xₜ with its previous hidden state hₜ₋₁ to generate its current hidden state hₜ; that is, hₜ=LSTM(xₜ, hₜ₋₁) for t=1 to t=T. The predictions for the three tasks are then performed with the corresponding hidden states as follows:

\hat{m}=sigmoid(Wₘh₄₈+bₘ)

\hat{d}ₜ=sigmoid(W_{d}hₜ+b_{d}) for t=5 . . . T

\hat{l}ₜ=softmax(Wₗhₜ+bₗ) for t=5 . . . T (1)

where \hat{m}, \hat{d}ₜ, and \hat{l}ₜ are the probabilities for in-hospital mortality, decompensation, and LOS, respectively, and Wₘ, W_{d}, and Wₗ are the respective weights of the fully-connected (FC) layer. Notice that the in-hospital mortality is predicted at end of 48 hours, while the predictions for decompensation and LOS tasks are done at each time step after first four hours of ICU stay. The models can be trained using cross entropy (CE) loss defined as below.

$${ℒ}_{ihm} = CE ( m , \hat{m} ) \\ {ℒ}_{decom} = \frac{1}{T} {\sum }_{t} CE ( {d}_{t} , {\hat{d}}_{t} ) \\ {ℒ}_{los} = \frac{1}{T} {\sum }_{t} CE ( {l}_{t} , {\hat{l}}_{t} ) \qquad ( 2 )$$

### Multi-Modal Neural Network

In the multimodal model, the goal is to improve the predictions by taking both the time series data xₜ and the doctor notes nᵢ as input to the network.

### Convolutional Feature Extractor for Doctor Notes.

As shown in FIG. 2, a convolutional approach can be used to extract the textual features from the doctor's notes. For a piece of clinical note N, the CNN takes the word embeddings e=(e₁, e₂, . . . , eₙ) as input and applies 1D convolution operations, followed by maxpooling over time to generate a p dimensional feature vector \hat{z}, which is fed to the fully connected layer alongside the LSTM output from time series signal (described in the next paragraph) for further processing. From now onwards, denote the 1D convolution over note N as \hat{z}=Conv1D(N).

### Model for In-Hospital Mortality.

This model takes the time series signals [xₜ]ₜ₌₁^{T} and all notes [Nᵢ]ᵢ₌₁^{K} to predict the mortality label m at t=T(T=48). For this, [xₜ]ₜ₌₁^{T} is processed through an LSTM layer as in the baseline model presented earlier, and for the notes, concatenate (⊗) all the notes N₁ to N_{K} charted between t=1 to t=T to generate a single document N_{T}. N48 represents concatenated notes until 48 hours, xₜ refers to time-series data at time t More formally,

N_{T}=N₁⊗N₂⊗ . . . ⊗N_{K}

hₜ=LSTM(xₜ,hₜ₋₁) for t=1 . . . T

\hat{z}=Conv1D(N_{T})

\hat{m}=sigmoid(W₁h₄₈+W₂\hat{z}+b) (3)

Using pre-trained word2vec embeddings trained on both MIMIC-Ill clinical notes and PubMed articles to initialize the methods as it outperforms other embeddings. Freeze the embedding layer parameters, since no improvements were observed by fine-tuning them.

### Model for Decompensation and Length of Stay.

Being sequential prediction problems, modeling decompensation and length-of-stay requires special technique to align the discrete text events to continuous time series signals, measured at 1 event per hour. Unlike in-hospital mortality, here extract feature maps zᵢ by processing each note Nᵢ independently using 1D convolution operations. For each time step t=1, 2 . . . T, let zₜ denote the extracted text feature map to be used for prediction at time step t. Here, nₜ and xₜ refers to notes and time-series data at time t. Compute zₜ as follows.

$${z}_{i} = Conv 1 D ( {N}_{i} ) for i = 1 … K \\ w ( t , i ) = exp [ - λ * ( t - CT ( i ) ) ] \\ {z}_{t} = \frac{1}{M} {\sum }_{i = 1}^{M} {z}_{i} w ( t , i ) \qquad ( 4 )$$

where M is the number of doctor notes seen before time-step t, and λ is a decay hyperparameter tuned on a validation data. Notice that zₜ is computed as a weighted sum of the feature vectors, where the weights are computed with an exponential decay function. A decay can give preference to recent notes as they better describe the current state of the patient.

The time series data xₜ is modeled using an LSTM as before. In one example, concatenate the attenuated output from the CNN with the LSTM output for the prediction tasks as follows:

hₜ=LSTM(xₜ,hₜ₋₁)

dₜ=sigmoid(W_{d}¹hₜ+W_{d}²zₜ+b)

\hat{l}ₜ=softmax(Wₗ¹hₜ+Wₗ²zₜ+b) (5)

Both the baselines and the multimodal networks are regularized using dropout and weight decay. In one example, an Adam Optimizer is used to train the models. Adam is an adaptive learning rate optimization algorithm designed for training deep neural networks. The algorithm uses adaptive learning rate methods to find individual learning rates for each parameter.

An experiment can be conducted using the MIMIC-III dataset following benchmark setup for processing the time series signals from ICU instruments. One example uses the same test-set defined in the benchmark and 15% of remaining data as validation set. For the in-hospital mortality task, only those patients are considered who were admitted in the ICU for at least 48 hours. Clinical notes without an associated chart time are omitted. Patients without clinical notes are omitted. Notes which have been charted before ICU admission are concatenated and treated as one note at t=1. In one experiment, after pre-processing, the number of patients for in-hospital mortality is 11,579 and 22,353 for the other two tasks.

For the in-hospital mortality task, best performing baseline and multimodal network have 256 hidden units LSTM cell. For convolution operation, one example uses 256 filters for each of kernel size 2, 3 and 4. For decompensation and LOS prediction, one example uses 64 hidden units for LSTM and 128 filters for each 2, 3 and 4 size convolution filters. In one example, the best decay factor A for text features was 0.01. Machine learning platform TensorFlow can be used for implementing some of the methods described herein. In one example, the models can be regularized using 0.2 dropout and 0.01 weight decay coefficient. Data shown here corresponds to five runs of an experiment with different initialization and report the mean and standard deviations.

Results can be analyzed using Area Under Precision-Recall (AUCPR) metric for in-hospital mortality and decompensation tasks as they suffer from class imbalance with only 10% patients suffering mortality, following the benchmark. AUCPR can yield good results for such an imbalanced class problem. Cohen's linear weighted kappa, which measures the correlation between predicted and actual multi-class buckets can be used to evaluate LOS.

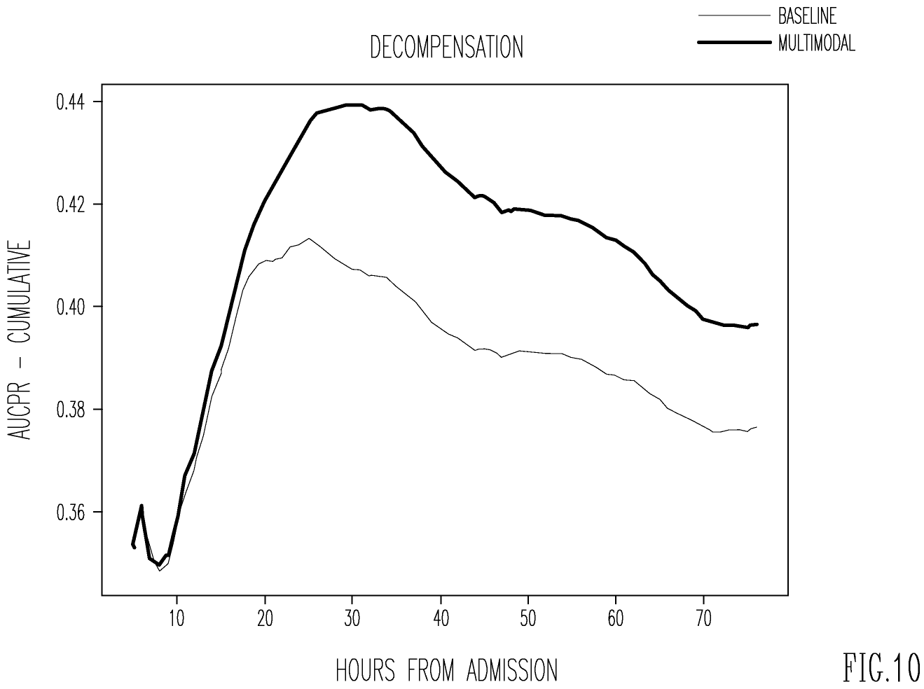

One example includes a comparison of multimodal network with the baseline time series LSTM models for all three tasks. Sample experimental results are shown in Tables 1A, 1B, and 1C. Graphical data for decompensation is shown in FIG. 10.

The multimodal network outperforms the time-series models for these three tasks. For in-hospital mortality prediction, the results show an improvement of around 7.8% over the baseline time series LSTM model. With the multimodal network, the results here shown an improvement of around 6% (see FIG. 10) and 3.5% for decompensation and LOS, respectively.

The data do not show a change in performance with respect to results reported in benchmark study despite dropping patients with no notes or chart time. In order to understand the predictive power of clinical notes, one example includes training text only models using CNN part from the model. In one example, average word embedding without CNN is used as another method to extract feature from the text as a baseline. Text-only-models perform poorly compared to time-series baseline. Hence, text can only provide additional predictive power on top of time-series data.

TABLE 1A
In-Hospital Mortality

| | AUCROC | AUCPR |
|---|---|---|
| Baseline (no text) | 0.844 | 0.487 |
| Text only | 0.793 | 0.303 |
| Multimodal - avg WE | 0.851 | 0.492 |
| Multimodal - IDCNN | 0.865 | 0.525 |

TABLE 1B
Decompensation

| | AUCROC | AUCPR |
|---|---|---|
| Baseline (no text) | 0.892 | 0.325 |
| Text only | 0.789 | 0.081 |
| Multimodal - avg WE | 0.902 | 0.311 |
| Multimodal - IDCNN | 0.907 | 0.345 |

TABLE 1C
Length of Stay

| | kappa |
|---|---|
| Baseline (no text) | 0.438 |
| Text only | 0.341 |
| Multimodal - avg WE | 0.449 |
| Multimodal - IDCNN | 0.453 |

Tables 1A, 1B, and 1C illustrate evaluated results for all three tasks. Standard deviations: IHM (AUCROC<0.004, AUCPR<0.015), Decompensation (AUCROC<0.008, AUCPR<0.008), and LOS (Kappa<0.003).

Early identification of a patient condition is critical for acute care and ICU management. Literature has exclusively focused on using time-series measurements from ICU instruments to this end. In one example of the present subject matter, using clinical notes along with time-series data can improve the prediction performance significantly.

### Machine Learning Embodiments

As discussed above, using artificial intelligence and/or machine learning techniques may be desirable for delivering better medical care and for improving management of medical facilities. Some aspects of the technology disclosed herein are directed to using artificial intelligence and/or machine learning techniques.

In some embodiments, a server generates and trains a deep neural network (DNN) model to improve health care outcomes. This can include developing a model or generating a prediction and providing that output to an edge device. The edge device. The edge device may be one or more of a desktop computer, a laptop computer, a tablet computer, a mobile phone, a digital music player, and a personal digital assistant (PDA).

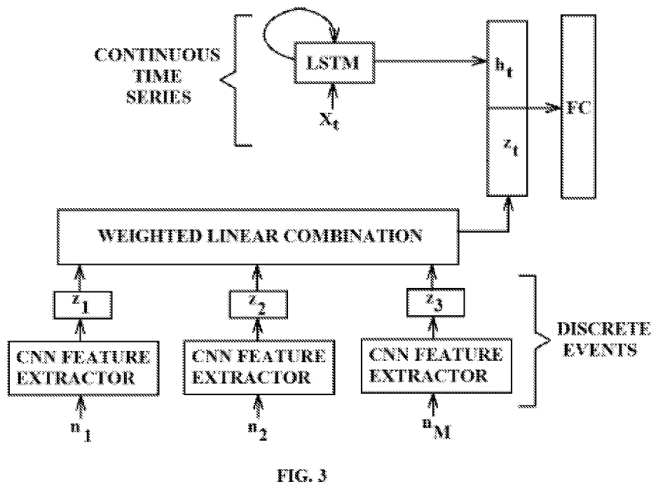

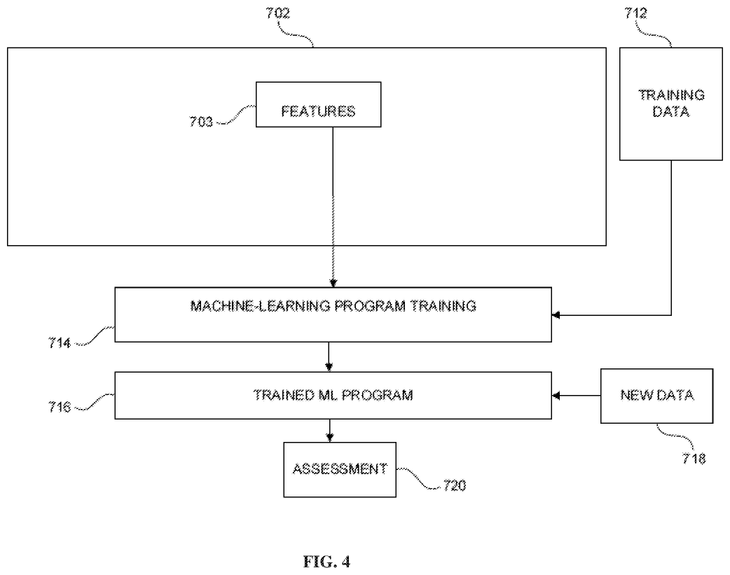

As used herein, the terms predict and manage encompasses their plain and ordinary meaning. Among other things, the term predict may refer to an artificial neural network (ANN) generating a measure of likelihood for an outcome. In addition, manage may refer to an administrative function concerning resources such as equipment and personnel involved in delivery of medical care. In the training phase of a supervised learning engine, human-generated input (or labels generated by another machine learning engine) are provided to the untrained or partially-trained ANN in order for the ANN to train itself to generate outputs, as described herein, for example, in conjunction with FIGS. 1-3.

Aspects of the systems and methods described herein may be implemented as part of a computer system. The computer system may be one physical machine, or may be distributed among multiple physical machines, such as by role or function, or by process thread in the case of a cloud computing distributed model. In various embodiments, aspects of the systems and methods described herein may be configured to run on desktop computers, embedded devices, mobile phones, physical server machines and in virtual machines that in turn are executed on one or more physical machines. It will be understood that features of the systems and methods described herein may be realized by a variety of different suitable machine implementations.

The system includes various engines, each of which is constructed, programmed, configured, or otherwise adapted, to carry out a function or set of functions. The term engine as used herein means a tangible device, component, or arrangement of components implemented using hardware, such as by an application specific integrated circuit (ASIC) or field-programmable gate array (FPGA), for example, or as a combination of hardware and software, such as by a processor-based computing platform and a set of program instructions that transform the computing platform into a special-purpose device to implement the particular functionality. An engine may also be implemented as a combination of the two, with certain functions facilitated by hardware alone, and other functions facilitated by a combination of hardware and software.

In an example, the software may reside in executable or non-executable form on a tangible machine-readable storage medium. Software residing in non-executable form may be compiled, translated, or otherwise converted to an executable form prior to, or during, runtime. In an example, the software, when executed by the underlying hardware of the engine, causes the hardware to perform the specified operations. Accordingly, an engine is physically constructed, or specifically configured (e.g., hardwired), or temporarily configured (e.g., programmed) to operate in a specified manner or to perform part or all of any operations described herein in connection with that engine.

Considering examples in which engines are temporarily configured, each of the engines may be instantiated at different moments in time. For example, where the engines comprise a general-purpose hardware processor core configured using software; the general-purpose hardware processor core may be configured as respective different engines at different times. Software may accordingly configure a hardware processor core, for example, to constitute a particular engine at one instance of time and to constitute a different engine at a different instance of time.

In certain implementations, at least a portion, and in some cases, all, of an engine may be executed on the processor(s) of one or more computers that execute an operating system, system programs, and application programs, while also implementing the engine using multitasking, multithreading, distributed (e.g., cluster, peer-peer, cloud, etc.) processing where appropriate, or other such techniques. Accordingly, each engine may be realized in a variety of suitable configurations, and should generally not be limited to any particular implementation exemplified herein, unless such limitations are expressly called out.

In addition, an engine may itself be composed of more than one sub-engines, each of which may be regarded as an engine in its own right. Moreover, in the embodiments described herein, each of the various engines corresponds to a defined functionality. However, it should be understood that in other contemplated embodiments, each functionality may be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

As used herein, the term “convolutional neural network” or “CNN” may refer, among other things, to a neural network that is comprised of one or more convolutional layers (often with a subsampling operation) and then followed by one or more fully connected layers as in a standard multilayer neural network. In some cases, the architecture of a CNN is designed to take advantage of the 2D structure of an input image. This is achieved with local connections and tied weights followed by some form of pooling which results in translation invariant features. In some cases, CNNs are easier to train and have many fewer parameters than fully connected networks with the same number of hidden units. In some embodiments, a CNN includes multiple hidden layers and, therefore, may be referred to as a deep neural network (DNN). CNNs are generally described in “ImageNet Classification with Deep Convolutional Neural Networks,” part of “Advances in Neural Information Processing Systems 25” (NIPS 2012) by Alex Krizhevsky, Ilya Sutskever, and Geoffrey E. Hinton, available at: papers.nips.cc/paper/4824-imagenet-classification-with-deep-convolutional-neural-networ, last visited 28 Aug. 2019, the entire content of which is incorporated herein by reference.

As used herein, the phrase “computing machine” encompasses its plain and ordinary meaning. A computing machine may include, among other things, a single machine with a processor and a memory or multiple machines that have access to one or more processors or one or more memories, sequentially or in parallel. A server may be a computing machine. A client device may be a computing machine. An edge device may be a computing machine. A data repository may be a computing machine.

Throughout this document, some method(s) are described as being implemented serially and in a given order. However, unless explicitly stated otherwise, the operations of the method(s) may be performed in any order. In some cases, two or more operations of the method(s) may be performed in parallel using any known parallel processing techniques. In some cases, some of the operation(s) may be skipped and/or replaced with other operations. Furthermore, skilled persons in the relevant art may recognize other operation(s) that may be performed in conjunction with the operation(s) of the method(s) disclosed herein.

FIG. 4 illustrates the training and use of a machine-learning program, according to some example embodiments. In some example embodiments, machine-learning programs (MLPs), also referred to as machine-learning algorithms or tools, are utilized to perform operations associated with machine learning tasks, such as optical character recognition or machine translation.

Machine learning (ML) is a field of study that gives computers the ability to learn without being explicitly programmed. Machine learning explores the study and construction of algorithms, also referred to herein as tools, which may learn from existing data and make predictions about new data. Such machine-learning tools operate by building a model from example training data 712 in order to make data-driven predictions or decisions expressed as outputs or assessments 720. Although example embodiments are presented with respect to a few machine-learning tools, the principles presented herein may be applied to other machine-learning tools.

In some example embodiments, different machine-learning tools may be used. For example, Logistic Regression (LR), Naive-Bayes, Random Forest (RF), neural networks (NN), matrix factorization, and Support Vector Machines (SVM) tools may be used for generating an output.

Two common types of problems in machine learning are classification problems and regression problems. Classification problems, also referred to as categorization problems, aim at classifying items into one of several category values (for example, is this object an apple or an orange). Regression algorithms aim at quantifying some items (for example, by providing a value that is a real number). The machine-learning algorithms utilize the training data 712 to find correlations among identified features 702 that affect the outcome.

The machine-learning algorithms utilize features 703 for analyzing the data to generate assessments 720. A feature 703 is an individual measurable property of a phenomenon being observed. The concept of a feature is related to that of an explanatory variable used in statistical techniques such as linear regression. Choosing informative, discriminating, and independent features is important for effective operation of the MLP in pattern recognition, classification, and regression. Features may be of different types, such as numeric features, strings, and graphs.

In one example embodiment, the features 703 may be of different types and may include various data features 703 that are detectable by a machine accessing an input. The features 703 may include numeric values, qualitative data, images, text, graphs, and the like.

The machine-learning algorithms utilize the training data 712 to find correlations among the identified features 702 that affect the outcome or assessment 720. In some example embodiments, the training data 712 includes labeled data, which is known data for one or more identified features 702 and one or more outcomes.

With the training data 712 and the identified features 702, the machine-learning tool is trained at operation 714. The machine-learning tool appraises the value of the features 702 as they correlate to the training data 712. The result of the training is the trained machine-learning program 716.

When the machine-learning program 716 is used to perform an assessment, new data 718 is provided as an input to the trained machine-learning program 716, and the machine-learning program 716 generates the assessment 720 as output.

Machine learning techniques train models to accurately make predictions on data fed into the models (e.g., patient morbidity, hospitalization stay duration, decompensation). During a learning phase, the models are developed against a training dataset of inputs to optimize the models to correctly predict the output for a given input. Generally, the learning phase may be supervised, semi-supervised, or unsupervised, indicating a decreasing level to which the “correct” outputs are provided in correspondence to the training inputs. In a supervised learning phase, all of the outputs are provided to the model and the model is directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set is provided, with some of the outputs known and some unknown for the training dataset.

Models may be run against a training dataset for several epochs (e.g., iterations), in which the training dataset is repeatedly fed into the model to refine its results. For example, in a supervised learning phase, a model is developed to predict the output for a given set of inputs and is evaluated over several epochs to more reliably provide the output that is specified as corresponding to the given input for the greatest number of inputs for the training dataset. In another example, for an unsupervised learning phase, a model is developed to cluster the dataset into n groups and is evaluated over several epochs as to how consistently it places a given input into a given group and how reliably it produces the n desired clusters across each epoch.

Once an epoch is run, the models are evaluated, and the values of their variables are adjusted to attempt to better refine the model in an iterative fashion. In various aspects, the evaluations are biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several other machine learning algorithms that may be applied with the present disclosure, including linear regression, random forests, decision tree learning, neural networks, deep neural networks, etc.

Each model develops a rule or algorithm over several epochs by varying the values of one or more variables affecting the inputs to more closely map to a desired result, but as the training dataset may be varied, and is preferably very large, perfect accuracy and precision may not be achievable. A number of epochs that make up a learning phase, therefore, may be set as a given number of trials or a fixed time/computing budget, or may be terminated before that number/budget is reached when the accuracy of a given model is high enough or low enough or an accuracy plateau has been reached. For example, if the training phase is designed to run n epochs and produce a model with at least 95% accuracy, and such a model is produced before the nth epoch, the learning phase may end early and use the produced model satisfying the end-goal accuracy threshold. Similarly, if a given model is inaccurate enough to satisfy a random chance threshold (e.g., the model is only 55% accurate in determining true/false outputs (or outputs for given inputs), the learning phase for that model may be terminated early, although other models in the learning phase may continue training. Similarly, when a given model continues to provide similar accuracy or vacillate in its results across multiple epochs—having reached a performance plateau—the learning phase for the given model may terminate before the epoch number/computing budget is reached.

Once the learning phase is complete, the models are finalized. In some example embodiments, models that are finalized are evaluated against testing criteria. In a first example, a testing dataset that includes known outputs for its inputs is fed into the finalized models to determine an accuracy of the model in handling data that it has not been trained on. In a second example, a false positive rate or false negative rate may be used to evaluate the models after finalization. In a third example, a delineation between data clusterings is used to select a model that produces the clearest bounds for its clusters of data.

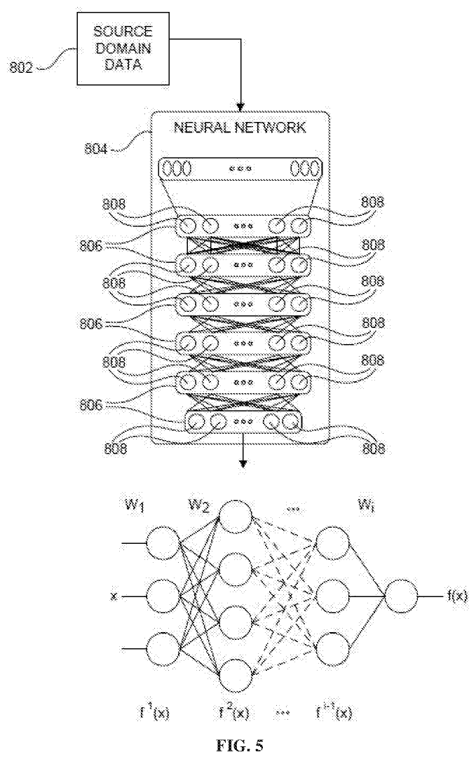

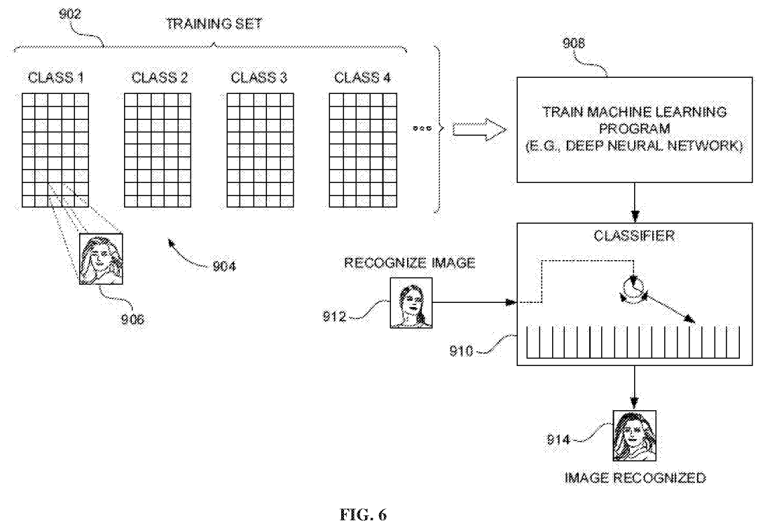

FIG. 5 illustrates an example neural network 804, in accordance with some embodiments. As shown, the neural network 804 receives, as input, source domain data 802. The input is passed through a plurality of layers 806 to arrive at an output. Each layer 806 includes multiple neurons 808. The neurons 808 receive input from neurons of a previous layer and apply weights to the values received from those neurons in order to generate a neuron output. The neuron outputs from the final layer 806 are combined to generate the output of the neural network 804.

As illustrated at the bottom of FIG. 5, the input is a vector x. The input is passed through multiple layers 806, where weights W1, W2, . . . , Wi are applied to the input to each layer to arrive at f1(x), f2(x), . . . , fi−1(x), until finally the output f(x) is computed. The weights are established (or adjusted) through learning and training of the network. As shown, each of the weights W1, W2, . . . , Wi is a vector. However, in some embodiments, one or more of the weights may be a scalar.

Neural networks utilize features for analyzing the data to generate assessments. A feature is an individual measurable property of a phenomenon being observed. The concept of feature is related to that of an explanatory variable used in statistical techniques such as linear regression. Further, deep features represent the output of nodes in hidden layers of the deep neural network.

A neural network, sometimes referred to as an artificial neural network, is a computing system/apparatus based on consideration of neural networks of biological brains. Such systems/apparatus progressively improve performance, which is referred to as learning, to perform tasks, typically without task-specific programming. For example, in image recognition, a neural network may be taught to identify images that contain an object by analyzing example images that have been tagged with a name for the object and, having learned the object and name, may use the analytic results to identify the object in untagged images. A neural network is based on a collection of connected units called neurons, where each connection, called a synapse, between neurons can transmit a unidirectional signal with an activating strength (e.g., a weight) that varies with the strength of the connection. The weight applied for the output of a first neuron at the input of a second neuron may correspond to the activating strength. The receiving neuron can activate and propagate a signal to downstream neurons connected to it, typically based on whether the combined incoming signals, which are from potentially many transmitting neurons, are of sufficient strength, where strength is a parameter.

A deep neural network (DNN) is a stacked neural network, which is composed of multiple layers. The layers are composed of nodes, which are locations where computation occurs, loosely patterned on a neuron in the biological brain, which fires when it encounters sufficient stimuli. A node combines input from the data with a set of coefficients, or weights, that either amplify or dampen that input, which assigns significance to inputs for the task the algorithm is trying to learn. These input-weight products are summed, and the sum is passed through what is called a node's activation function, to determine whether and to what extent that signal progresses further through the network to affect the ultimate outcome. A DNN uses a cascade of many layers of non-linear processing units for feature extraction and transformation. Each successive layer uses the output from the previous layer as input. Higher-level features are derived from lower-level features to form a hierarchical representation. The layers following the input layer may be convolution layers that produce feature maps that are filtering results of the inputs and are used by the next convolution layer.

In training of a DNN architecture, a regression, which is structured as a set of statistical processes for estimating the relationships among variables, can include a minimization of a cost function. The cost function may be implemented as a function to return a number representing how well the neural network performed in mapping training examples to correct output. In training, if the cost function value is not within a pre-determined range, based on the known training images, backpropagation is used, where backpropagation is a common method of training artificial neural networks that are used with an optimization method such as a stochastic gradient descent (SGD) method.

Use of backpropagation can include propagation and weight update. When an input is presented to the neural network, it is propagated forward through the neural network, layer by layer, until it reaches the output layer. The output of the neural network is then compared to the desired output, using the cost function, and an error value is calculated for each of the nodes in the output layer. The error values are propagated backwards, starting from the output, until each node has an associated error value which roughly represents its contribution to the original output. Backpropagation can use these error values to calculate the gradient of the cost function with respect to the weights in the neural network. The calculated gradient is fed to the selected optimization method to update the weights to attempt to minimize the cost function.

FIG. 6 illustrates the training of an image recognition machine learning program, in accordance with some embodiments. The machine learning program may be implemented at one or more computing machines. Block 902 illustrates a training set, which includes multiple classes 904. Each class 904 includes multiple images 906 associated with the class. Each class 904 may correspond to a type of object in the image 906 (e.g., a digit 0-9, a man or a woman, a cat or a dog). In one example, the machine learning program is trained to recognize images of the presidents of the United States, and each class corresponds to each president (e.g., one class corresponds to Barack Obama, one class corresponds to George W. Bush, one class corresponds to Bill Clinton, etc.). At block 908 the machine learning program is trained, for example, using a deep neural network. At block 910, the trained classifier, generated by the training of block 908, recognizes an image 912, and at block 914 the image is recognized. For example, if the image 912 is a photograph of Bill Clinton, the classifier recognizes the image as corresponding to Bill Clinton at block 914.

FIG. 6 illustrates the training of a classifier, according to some example embodiments. A machine learning algorithm is designed for recognizing faces, and a training set 902 includes data that maps a sample to a class 904 (e.g., a class includes all the images of purses). The classes may also be referred to as labels. Although embodiments presented herein are presented with reference to object recognition, the same principles may be applied to train machine-learning programs used for recognizing any type of items.

The training set 902 includes a plurality of images 906 for each class 904 (e.g., image 906), and each image is associated with one of the categories to be recognized (e.g., a class). The machine learning program is trained 908 with the training data to generate a classifier 910 operable to recognize images. In some example embodiments, the machine learning program is a DNN.

When an input image 912 is to be recognized, the classifier 910 analyzes the input image 912 to identify the class (e.g., class 914) corresponding to the input image 912.

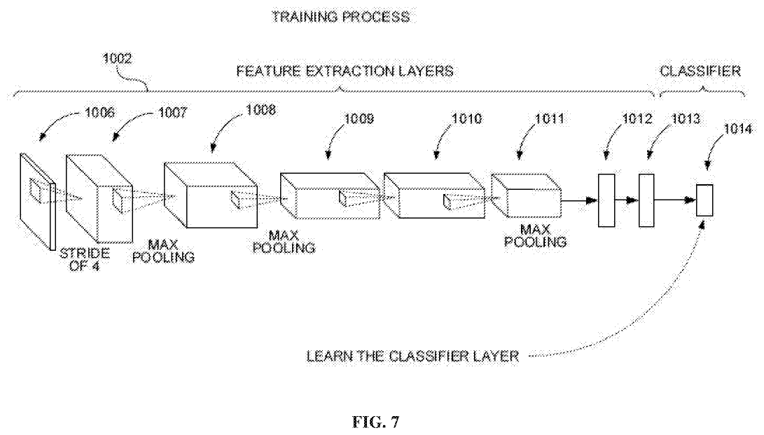

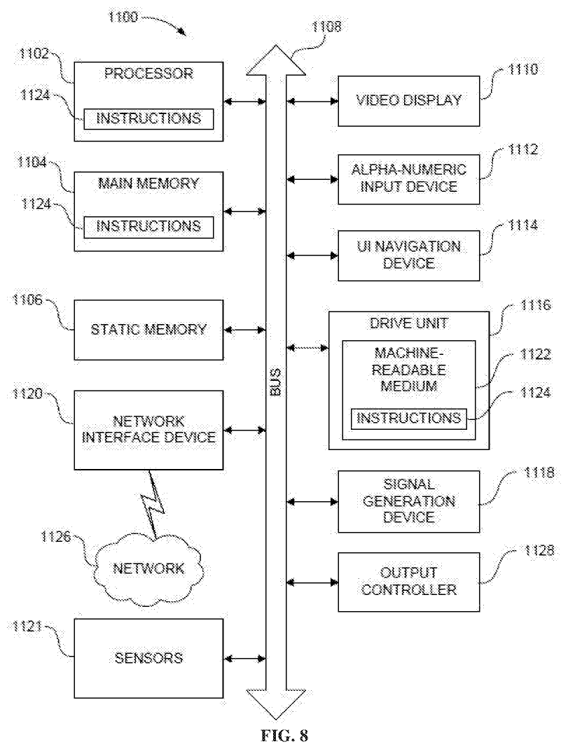

FIG. 7 illustrates the feature-extraction process and classifier training, according to some example embodiments. Training the classifier may be divided into feature extraction layers 1002 and classifier layer 1014. Each image is analyzed in sequence by a plurality of layers 1006-1013 in the feature-extraction layers 1002. As discussed below, some embodiments of machine learning are used for facial classification (i.e., classifying a given facial image as belonging to a given person, such as Barack Obama, George W. Bush, Bill Clinton, the owner of a given mobile phone, and the like). However, as discussed herein, a facial recognition image classification neural network or a general image classification neural network (that classifies an image as including a given object, such as a table, a chair, a lamp, and the like) may be further trained to make predictions or manage a health care facility.

With the development of deep convolutional neural networks, the focus in face recognition has been to learn a good face feature space, in which faces of the same person are close to each other and faces of different persons are far away from each other. For example, the verification task with the LFW (Labeled Faces in the Wild) dataset has been often used for face verification.

Many face identification datasets (e.g., MegaFace and LFW) that are used for face identification tasks are based on a similarity comparison between the images in the gallery set and the query set, which is essentially a K-nearest-neighborhood (KNN) method to estimate the person's identity. In the ideal case, there is a good face feature extractor (inter-class distance is always larger than the intra-class distance), and the KNN method is adequate to estimate the person's identity.

Feature extraction is a process to reduce the amount of resources required to describe a large set of data. When performing analysis of complex data, one of the major problems stems from the number of variables involved. Analysis with a large number of variables generally uses a large amount of memory and computational power, and it may cause a classification algorithm to overfit to training samples and generalize poorly to new samples. Feature extraction is a general term describing methods of constructing combinations of variables to get around these large data-set problems while still describing the data with sufficient accuracy for the desired purpose.

In some example embodiments, feature extraction starts from an initial set of measured data and builds derived values (features) intended to be informative and non-redundant, facilitating the subsequent learning and generalization operations. Further, feature extraction is related to dimensionality reduction, such as reducing large vectors (sometimes with very sparse data) to smaller vectors capturing the same, or similar, amount of information.

Determining a subset of the initial features is called feature selection. The selected features are expected to contain the relevant information from the input data, so that the desired task can be performed by using this reduced representation instead of the complete initial data. DNN utilizes a stack of layers, where each layer performs a function. For example, the layer could be a convolution, a non-linear transform, the calculation of an average, etc. Eventually this DNN produces outputs by classifier 1014. In FIG. 7, the data travels from left to right and the features are extracted. The goal of training the neural network is to find the weights for all the layers that make them adequate for the desired task.

As shown in FIG. 7, a “stride of 4” filter is applied at layer 1006, and max pooling is applied at layers 1007-1013. The stride controls how the filter convolves around the input volume. “Stride of 4” refers to the filter convolving around the input volume four units at a time. Max pooling refers to down-sampling by selecting the maximum value in each max pooled region.

In some example embodiments, the structure of each layer is predefined. For example, a convolution layer may contain small convolution kernels and their respective convolution parameters, and a summation layer may calculate the sum, or the weighted sum, of two pixels of the input image. Training assists in defining the weight coefficients for the summation.

One way to improve the performance of DNNs is to identify newer structures for the feature-extraction layers, and another way is by improving the way the weights are identified at the different layers for accomplishing a desired task. The challenge is that for a typical neural network, there may be millions of weights to be optimized. Trying to optimize all these weights from scratch may take hours, days, or even weeks, depending on the amount of computing resources available and the amount of data in the training set.

FIG. 8 illustrates a circuit block diagram of a computing machine 1100 in accordance with some embodiments. In some embodiments, components of the computing machine 1100 may store or be integrated into other components shown in the circuit block diagram of FIG. 8. For example, portions of the computing machine 1100 may reside in the processor 1102 and may be referred to as “processing circuitry.” Processing circuitry may include processing hardware, for example, one or more central processing units (CPUs), one or more graphics processing units (GPUs), and the like. In alternative embodiments, the computing machine 1100 may operate as a standalone device or may be connected (e.g., networked) to other computers. In a networked deployment, the computing machine 1100 may operate in the capacity of a server, a client, or both in server-client network environments. In an example, the computing machine 1100 may act as a peer machine in peer-to-peer (P2P) (or other distributed) network environment. The computing machine 1100 may be a specialized computer, a personal computer (PC), a tablet PC, a personal digital assistant (PDA), a mobile telephone, a smart phone, a web appliance, a network router, switch or bridge, or any machine capable of executing instructions (sequential or otherwise) that specify actions to be taken by that machine.

Examples, as described herein, may include, or may operate on, logic or a number of components, modules, or mechanisms. Modules and components are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems/apparatus (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” (and “component”) is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

The computing machine 1100 may include a hardware processor 1102 (e.g., a central processing unit (CPU), a GPU, a hardware processor core, or any combination thereof), a main memory 1104 and a static memory 1106, some or all of which may communicate with each other via an interlink (e.g., bus) 1108. Although not shown, the main memory 1104 may contain any or all of removable storage and non-removable storage, volatile memory or non-volatile memory. The computing machine 1100 may further include a video display unit 1110 (or other display unit), an alphanumeric input device 1112 (e.g., a keyboard), and a user interface (UI) navigation device 1114 (e.g., a mouse). In an example, the display unit 1110, input device 1112 and UI navigation device 1114 may be a touch screen display. The computing machine 1100 may additionally include a storage device (e.g., drive unit) 1116, a signal generation device 1118 (e.g., a speaker), a network interface device 1120, and one or more sensors 1121, such as a global positioning system (GPS) sensor, compass, accelerometer, or other sensor. The computing machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The drive unit 1116 (e.g., a storage device) may include a machine readable medium 1122 on which is stored one or more sets of data structures or instructions 1124 (e.g., software) embodying or utilized by any one or more of the techniques or functions described herein. The instructions 1124 may also reside, completely or at least partially, within the main memory 1104, within static memory 1106, or within the hardware processor 1102 during execution thereof by the computing machine 1100. In an example, one or any combination of the hardware processor 1102, the main memory 1104, the static memory 1106, or the storage device 1116 may constitute machine readable media.

While the machine readable medium 1122 is illustrated as a single medium, the term “machine readable medium” may include a single medium or multiple media (e.g., a centralized or distributed database, and/or associated caches and servers) configured to store the one or more instructions 1124.

The term “machine readable medium” may include any medium that is capable of storing, encoding, or carrying instructions for execution by the computing machine 1100 and that cause the computing machine 1100 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); and CD-ROM and DVD-ROM disks. In some examples, machine readable media may include non-transitory machine-readable media. In some examples, machine readable media may include machine readable media that is not a transitory propagating signal.

The instructions 1124 may further be transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, among others. In an example, the network interface device 1120 may include one or more physical jacks (e.g., Ethernet, coaxial, or phone jacks) or one or more antennas to connect to the communications network 1126.

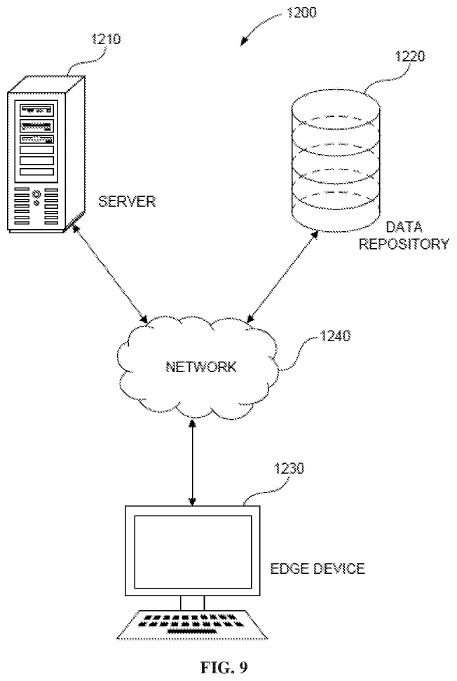

FIG. 9 illustrates an example system 1200 in which artificial intelligence-based yarn quality control may be implemented, in accordance with some embodiments. As shown, the system 1200 includes a server 1210, a data repository 1220, and an edge device 1230. The server 1210, the data repository 1220, and the edge device 1230 communicate with one another over a network 1240. The network 1240 may include one or more of the internet, an intranet, a local area network, a wide area network, a cellular network, a WiFi® network, a virtual private network, a wired network, a wireless network, and the like. In some embodiments, a direct wired or wireless connection may be used in addition to or in place of the network 1240.

The data repository 1220 stores data. The data can include monitoring data from medical care instrumentation. The data can include clinical notes from a doctor, caregiver, or other provider and may be generated at the server 1210 as described herein. The edge device 1230 may be one or more of a desktop computer, a laptop computer, a tablet computer, a mobile phone, a digital music player, and a personal digital assistant (PDA). The server 1210 generates and trains a DNN model to make a prediction or to manage an element of the medical care facility. The DNN model may be a CNN model or any other type of DNN model. Examples of operation of the server 1210 are discussed herein.

In FIG. 9, the server 1210, the data repository 1220, and the edge device 1230 are illustrated as being separate machines. However, in some embodiments, a single machine may include two or more of the server 1210, the data repository 1220, and the edge device 1230. In some embodiments, the functions of the server 1210 may be split between two or more machines. In some embodiments, the functions of the data repository 1220 may be split between two or more machines. In some embodiments, the functions of the edge device 1230 may be split between two or more machines.

The server 1210 may store, train, and inference with a generative adversarial network (GAN), an image recognition DNN model, and a transfer learning engine. The GAN and the image recognition DNN model may be implemented as an engine using software, hardware or a combination of software and hardware.

In a GAN, two neural networks contest with each other in a game (in the sense of game theory, often but not always in the form of a zero-sum game). Given a training set, this technique learns to generate new data with the same statistics as the training set. For example, a GAN trained on photographs can generate new photographs that look at least superficially authentic to human observers, having many realistic characteristics. Though originally proposed as a form of generative model for unsupervised learning, GANs have also proven useful for semi-supervised learning, fully supervised learning, and reinforcement learning.

In some examples, the output associated with the probability may include the probability itself or a mathematical function of the probability. The output associated with the probability may include a first value (e.g., TRUE) if the probability is greater than a threshold (e.g., 50%, 70% or 90%) and a second value (e.g., FALSE) if the probability is less than the threshold.

### Various Notes

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A method implemented at one or more computing machines, the method comprising:
receiving, using a server, time-series data corresponding to monitoring instrumentation in a medical care facility, the time-series data corresponding to a selected care recipient, the time-series data stored in one or more data storage units, the time-series data comprising data correlated with a plurality of regular time intervals;
receiving, using a server, aperiodic data corresponding to clinical notes collected in the medical care facility and corresponding to the selected care recipient, the aperiodic data stored in one or more data storage units, the aperiodic data including a time stamp; and
generating, using a deep neural network and the time-series data and using a convolutional neural network (CNN) and the aperiodic data, a plurality of computer-generated data corresponding to management of the medical care facility or medical condition of the care recipient.

2. The method of claim 1, wherein generating the plurality of computer-generated data includes using aggregated word embeddings based on the clinical notes.

3. The method of claim 1, wherein generating the plurality of computer-generated data includes executing natural language processing.

4. The method of claim 1, wherein generating the plurality of computer-generated data includes generating a prediction.

5. The method of claim 4, wherein generating the prediction includes at least one of predicting in-hospital mortality, predicting decompensation, and predicting length of stay.

6. A machine-readable medium storing instructions which, when executed at one or more computing machines, cause the one or more computing machines to perform operations comprising:
receiving, using a server, periodic data corresponding to instrumentation in a medical care facility associated with a selected medical care recipient;
receiving using the server, aperiodic data corresponding to care-giver notes associated with the selected medical care recipient;
generating an output using machine learning, the output corresponding to at least one of a prediction associated with the selected medical care recipient and management of the medical care facility; and
providing the output.

7. The machine-readable medium of claim 6, wherein providing the output comprises providing the model to an edge device for deployment thereat, wherein the edge device comprises one or more of a desktop computer, a laptop computer, a tablet computer, a mobile phone, a digital music player, and a personal digital assistant (PDA).

8. The machine-readable medium of claim 6, wherein generating the output includes executing a convolutional neural network based on the care-giver notes.

9. The machine-readable medium of claim 6, wherein generating the output includes executing a recurrent neural network (RNN) based on the periodic data.

10. A system comprising:
processing circuitry; and
a memory storing instructions which, when executed at the processing circuitry, cause the processing circuitry to perform operations including receiving time-series data corresponding to instrumentation associated with a selected care-recipient in a medical facility, receiving aperiodic data corresponding to clinical notes associated with the selected care-recipient, and generating an output, where the output includes at least one of a prediction as to the selected care-recipient and management of the medical facility.
